(19) United States
(12) Patent Application Publication
(10) Pub. No.: US 2019/0192485 A1
(43) Pub. Date: Jun. 27, 2019

(54) DOSAGE REGIMEN FOR THE TREATMENT OF ENDOMETRIOSIS

(71) Applicant: MEREO BIOPHARMA 2 LIMITED, London (GB)

(72) Inventors: Jackie PARKIN, London (GB); Lloyd B. KLICKSTEIN, Cambridge, MA (US)

(73) Assignee: MEREO BIOPHARMA 2 LIMITED, London (GB)

(21) Appl. No.: 16/325,872

(22) PCT Filed: Aug. 21, 2017
(86) PCT No.: PCT/GB2017/052466

§ 371 Date: Feb. 15, 2019

(30) Foreign Application Priority Data
Aug. 19, 2016 (GB) 1614179.8

(51) Int. Cl. A61K 31/4196; A61K 31/567; A61P 15/18; A61K 9/00; A61K 9/48; A61P 15/02

(52) U.S. Cl. CPC A61K 31/4196; A61K 31/567; A61P 15/18; A61K 45/06; A61K 9/4866; A61P 15/02; A61K 9/0053

(57) ABSTRACT

This invention relates to a dosage regimen comprising 4,4′-[fluoro-(1-H-1,2,4-triazol-1-yl)methylene]bisbenzonitrile and a combined oral contraceptive for use in the treatment of endometriosis in a non-pregnant, pre-menopausal female. The invention also relates to a multi-phase combination preparation comprising 4,4′-[fluoro-(1-H-1,2,4-triazol-1-yl)methylene]bisbenzonitrile and a combined oral contraceptive. The invention also relates to a kit comprising said multi-phase preparation and instructions for use.

### BACKGROUND OF THE INVENTION

Endometriosis is defined as ectopic endometrial tissue, commonly associated with localized pain. The ectopic tissue is usually found on pelvic organs and tissues and probably delivered by retrograde movement of endometrial cells sloughed during menses through the fallopian tubes. Although endometriosis typically causes pain, which may be severe, it also may be associated with adhesions and secondary infertility.

Endometriosis is an estrogen-dependent disease. The current standard of care for endometriosis is initially combination oral contraceptive (COC) therapy, and if that fails a gonadotropin releasing hormone (GnRH) analog. There are two common features of all approved drugs for endometriosis: the first is inhibition of ovulation; the second is an inhibition of follicle stimulating hormone (FSH) and luteinizing hormone (LH), particularly with GnRH analogs leading to a down-regulation of the cytochrome enzymes involved in ovarian steroidogenesis. The associated decrease in blood estrogens and lack of follicular rupture reduce estrogenic drive to endometrial tissue.

However, in addition to the role of circulating estrogens under hypothalamic-pituitary and gonadal control, it has also been shown that there is estrogen synthesis in the ectopic endometrial tissue with autocrine growth stimulation of the endometriosis implants and associated symptoms (Bulun et al, “Benign Diseases of the Vulva and Vagina”, Adolescent and Pediatric Gynecology, Volume 3, Issue 2, Pages 63-123, 1990). There is an association of endometriosis with obesity and aromatisation of androgenic hormones to estrogens in peritoneal and pelvic fat may contribute to the 1000-fold higher levels of hormones, including estrogens in the pelvic fluid that bathes the endometrial deposits compared to blood (Koninckx, Kennedy and Barlow, “Pathogenesis of Endometriosis: The Role of Peritoneal Fluid”, Gynecologic and Obstetric Investigation, Volume 47, Suppl. 1, Pages 23-33, 1999). Estrogen production by endometrial tissue and local fat is not under hypothalamic-pituitary-gonadal control and therefore is not affected by COCs or GnRH analogs.

COCs are typically administered over a 28-day period, however may be administered for longer. The impeded androgen danazol is approved for endometriosis treatment; however virilizing side effects have substantially limited its use. At least 10% of patients currently have no therapeutic options.

The key enzyme involved in tissue estrogen production is aromatase. The aromatase enzyme catalyzes the conversion of androgens to estrogen, for example endogenous androstenedione to estrone and endogenous testosterone into estradiol. Aromatase inhibitors limit the action of the aromatase enzyme, thereby reducing estrogen levels in a patient.

Accordingly, an aromatase inhibitor, which blocks estrogen synthesis broadly, should be effective in all patients, including those who fail to respond to the standard treatments.

Consistent with the hypothesis of unregulated local estrogen synthesis in some patients with refractory endometriosis, several small academic studies have supported the concept that patients with endometriosis respond at least partially to currently marketed aromatase inhibitors co-administered with COC, a progestin or a GnRH analog (reviewed in Attar and Bulun, “Aromatase inhibitors: the next generation of therapeutics for endometriosis?”, Fertility and sterility, Volume 85, Issue 5, Pages 1307-1318, 2006). Aromatase inhibitors have, however, been related to teratogenic effects and should not be prescribed to pregnant women (Tiboni G M, Marotta F, Rossi C, and Giampietro F, “Effects of the aromatase inhibitor letrozole on in utero development in rats”, Human Reproduction, Volume 23, Issue 8, Pages 1719-1723, 2008).

In addition to the clinically approved non-steroidal aromatase inhibitors anastrozole, letrozole and fadrozole, which are approved for the treatment of hormone dependent breast cancer by daily administration of dosages in the mg range, several other aromatase inhibitors have been described in the patent and scientific literature. One of these compounds is the aromatase inhibitor 4,4′-[fluoro-(1-H-1,2,4-triazol-1-yl)methylene]bisbenzonitrile, also known as 4-[α-4-cyanophenyl)-α-fluoro-1-(1,2,4-triazolyl)methyl]-benzonitrile or CGP47645, first described in 1992 [EP 490 816 and U.S. Pat. No. 5,637,605], having the following structural formula:

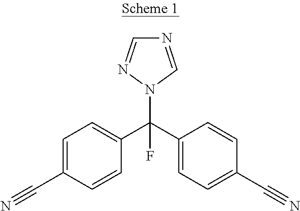 [SMILES: N#Cc1ccc(C(F)(c2ccc(C#N)cc2)n2cncn2)cc1]

CGP47645 is an aromatase inhibitor that is structurally related to letrozole, bearing a single fluorine-for-hydrogen substitution, with a prolonged duration of action.

Administration of CGP47645, a highly selective aromatase inhibitor, shows dose dependent reduction of the conversion of testosterone to estrone, estrone sulfate and estradiol.

Preliminary in-vitro and in-vivo experiments with this compound in rats and monkeys showed a similar up to 10 fold higher potency of aromatase inhibition than letrozole, and demonstrated the potential for a less than daily treatment regimen. A once weekly administration of 3 mg/kg of CGP47645 was considered as an effective dose achieving medical castration in adult female rats (Batzl-Hartmann et al, “Pharmacological profile of CGP47645, a new non-steroidal aromatase inhibitor with a long duration of action”, XVI International Cancer Congress, Pages 3041-3047, 1994). It was concluded that the half-life of CGP47645 is long enough to maintain endocrine efficacy similar to that of ovariectomy with a once-weekly dosing schedule (Bhatnagar et al, “Pharmacology of nonsteroidal aromatase inhibitors”, Hormone-dependent cancer, Pages 155-168, 1996).

Dosing of CGP47645 was surprisingly found to inhibit conversion of androgens to estrogens in man at doses significantly lower than that predicted by the blood PK and IC₅₀s. Further investigation demonstrated that tissue (fat) biopsies showed up to 10-fold higher concentration of CGP47645 than in plasma. The targeting of CGP47645 to the sites of aromatase activation in endometriosis is proposed to support effective suppression with doses that have low systemic activity with safety benefit.

Low doses of CGP47645 have the additional benefit of a shorter half-life to support the control of systemic exposure in women of child bearing potential in regimes with combined oral contraceptives.

### SUMMARY OF THE INVENTION

A dosage regimen comprising 4,4′-[fluoro-(1-H-1,2,4-triazol-1-yl)methylene]bisbenzonitrile and a combined oral contraceptive for use in the treatment of endometriosis in a non-pregnant, pre-menopausal female, wherein the 4,4′-[fluoro-(1-H-1,2,4-triazol-1-yl)methylene]bisbenzonitrile is administered in doses of about 0.001 mg to about 0.1 mg, wherein the combined oral contraceptive is administered once daily for an n[28-day] cycle of treatment, wherein n is a positive integer multiplier, wherein n is between 1 and 3 inclusive, and wherein the 4,4′-[fluoro-(1-H-1,2,4-triazol-1-yl)methylene]bisbenzonitrile is administered once every day, once every other day, once every 7 days or once every 28 days during the n[28-day] cycle of treatment.

Preferably, the 4,4′-[fluoro-(1-H-1,2,4-triazol-1-yl)methylene]bisbenzonitrile is administered once every day during the n[28-day] cycle of treatment for doses of about 0.001 mg to about 0.004 mg, more preferably about 0.001 mg to about 0.002 mg. Preferably, the 4,4′-[fluoro-(1-H-1,2,4-triazol-1-yl)methylene]bisbenzonitrile is administered once every day to include administration on days 1-7 up to Days 1-21, preferably days 1-14 of each menstrual cycle.

Preferably, the 4,4′-[fluoro-(1-H-1,2,4-triazol-1-yl)methylene]bisbenzonitrile is administered once every other day during the n[28-day] cycle of treatment for doses of about 0.004 mg to about 0.009 mg, preferably about 0.004 mg to about 0.006 mg. Preferably, the 4,4′-[fluoro-(1-H-1,2,4-triazol-1-yl)methylene]bisbenzonitrile is administered once every other day, to include administration on days 1-6 up to days 1-20, preferably days 1-14 of each menstrual cycle.

Preferably, the 4,4′-[fluoro-(1-H-1,2,4-triazol-1-yl)methylene]bisbenzonitrile is administered once every 7 days during the n[28-day] cycle of treatment for doses of about 0.009 mg to about 0.05 mg, more preferably about 0.009 mg to about 0.03 mg. Preferably, the 4,4′-[fluoro-(1-H-1,2,4-triazol-1-yl)methylene]bisbenzonitrile is administered once every 7 days, to include administration on days 1 to day 14, preferable day 1 and day 7, of each menstrual cycle.

Preferably, the 4,4′-[fluoro-(1-H-1,2,4-triazol-1-yl)methylene]bisbenzonitrile is administered once every 28 days during the n[28-day] cycle of treatment for doses of about 0.05 mg to about 0.1 mg, more preferably about 0.05 mg to about 0.075 mg.

A multi-phase combination preparation comprising one of the dosage regimens described above, wherein the multi-phase combination preparation comprises n[28-day] dosage units consistent with the dosage regimen such that each dosage unit comprises the 4,4′-[fluoro-(1-H-1,2,4-triazol-1-yl)methylene]bisbenzonitrile and combined oral contraceptive in a combined form, separately, or the combined oral contraceptive alone.

A kit comprising the multi-phase preparation described above, wherein the kit further comprises instructions on how to administer the dosage regimen for the treatment of a non-pregnant, pre-menopausal female with endometriosis.

### BRIEF DESCRIPTION OF THE FIGURES

FIG. 1: Testosterone levels in obese men associated with hypogonadotropic hypogonadism with low testosterone levels after doses of CGP47645 commencing 0.003 mg to 0.01 mg.

FIG. 2: Arithmetic mean (SD) plasma concentration time profile for 30 microgram Ethinyl Estradiol at steady state when administered with a single dose of placebo or 1 mg CGP47645 (log-linear plot); ▾: N=14, Δ: N=15.

FIG. 3: Arithmetic mean (SD) plasma concentration time profile for 0.15 mg Levonorgestrel at steady state when administered with a single dose of placebo or 1 mg CGP47645 (log-linear plot); [symbol] : N=14: [symbol] : N=15

### ABBREVIATIONS

Throughout this specification, the following abbreviations will be used:

AUC area under the concentration time curve

COC combined oral contraceptive

DS drug substance

GnRH gonadotrophic hormone releasing hormone

LH luteinizing hormone

LLOQ lower limit of quantification

mL millilitre(s)

MTD minimal toxic dose

OHH obese hypogonadotropic hypogonadal

PD pharmacodynamics

PK pharmacokinetics

### DEFINITIONS

Throughout this specification and in the claims that follow, the following terms are defined with the following meanings, unless explicitly stated otherwise.

As used herein, the terms “comprising” and “including” are used herein in their open, non-limiting sense.

Where the plural form is used for compounds, salts, and the like, this is taken to mean also a single compound, salt, or the like.

The term “aromatase inhibitor” is defined as a compound that inhibits the enzyme aromatase.

The term “compound” shall here be understood to cover any and all isomers (e.g., enantiomers, stereoisomers, diastereomers, rotomers, tautomers) or any mixture of isomers, prodrugs, and any pharmaceutically acceptable addition salts of 4,4′-[fluoro-(1-H-1,2,4-triazol-1-yl)methylene]bisbenzonitrile, unless stated otherwise.

As used herein, the term “elimination half-life” of a drug refers to the time required for the concentration of the drug in serum or plasma, to decrease by half, in vivo, for example due to degradation and/or clearance or sequestration by natural mechanisms. When determined experimentally by measuring drug concentration in plasma samples drawn at various and successive times after drug intake, this parameter is named “apparent elimination half-life”, designated T½. Methods for pharmacokinetic analysis and determination of drug half-life will be familiar to those skilled in the art, Pharmacokinetic parameters such as “apparent elimination half-life” T½ and area under the curve (AUC) can be determined from a curve of plasma or serum concentration of the drug against time. In particular, the following pharmacokinetic definitions shall apply:

AUC₀₋ₜ the AUC from time zero to time ‘t’, where t is the last sampling time point [mass×time×volume⁻¹]

AUC_{0-∞} the AUC from time zero to infinity [mass×time×volume−1]

Cₘₐₓ the maximum (peak) observed plasma, blood, serum, or other body fluid drug concentration after single dose administration [mass×volume⁻¹]

Cₗₐₛₜ the last measurable plasma, blood, serum, or other body fluid drug concentration

CL the total body clearance of drug from the plasma [volume×time⁻¹]
Clearance values from other body fluids may be noted by use of proper subscripts, for example CL_{b} refers to clearance from the blood and CLᵤ clearance of unbound drug from the plasma. If the clearance is following extravascular dose and bioavailability parameter is not known, then the notation should be CL/F

t time after drug administration [time]

T time last measurable concentration (when Cₗₐₛₜ occurs)

Tₘₐₓ the time to reach maximum (peak) plasma, blood, serum, or other body fluid drug concentration after single dose administration [time]

T_{1/2} the elimination half-life associated with the terminal slope (λ_{z}) of a semi logarithmic concentration-time curve [time]

The drug concentration in plasma and/or serum samples can be determined by a number of different ways, e.g. HPLC or LC-MS/MS analyses. In one embodiment, the concentration of 4,4′-[fluoro-(1-H-1,2,4-triazol-1-yl)methylene]bisbenzonitrile in plasma is analyzed using a validated LC-MS/MS method with a lower limit of quantification (LLOQ) at 0.1 ng/mL or better. In another embodiment, the concentration of 4,4′-[fluoro-(1-H-1,2,4-triazol-1-yl)methylene]bisbenzonitrile in human plasma is analyzed using a validated LC-MS/MS method with a lower limit of quantification (LLOQ) at 0.025 ng/mL.

As used herein, the term combined oral contraceptive (COC) is used to describe any pharmaceutically acceptable combined oral contraceptive which contains both synthetic estrogens and progestogens.

As used herein, the term monophasic COC is used to describe a COC which offers the same dose of synthetic estrogens and progestogens in each COC pill.

As used herein, the term multiphasic COC is used to describe a COC which offer varying doses of synthetic estrogens and progestogens in each COC pill.

As used herein, the term simultaneous dosing is used to describe an administration method whereby the drug formulations are taken concurrently for a given day in the dosage regimen (i.e. a 4,4′-[fluoro-(1-H-1,2,4-triazol-1-yl)methylene]bisbenzonitrile formulation and a COC formulation are taken at the same time).

As used herein, the term sequential dosing is used to describe an administration method whereby each drug formulation is administered individually, but immediately proceeded by the administration of the next drug for a given day in the dosage regimen (i.e. a 4,4′-[fluoro-(1-H-1,2,4-triazol-1-yl)methylene]bisbenzonitrile formulation is taken first, then a COC formulation is taken immediately after, or vice versa).

As used herein, the start of the menstrual cycle is defined as the last day of menstrual bleeding, or for those on COC, the last day of withdrawal bleeding or the last day of monthly COC dose.

### DETAILED DESCRIPTION OF THE INVENTION

According to the present invention, low doses of the aromatase inhibitor compound 4,4′-[fluoro-(1-H-1,2,4-triazol-1-yl)methylene]bisbenzonitrile are used in combination with a COC for the treatment of endometriosis in a non-pregnant, pre-menopausal female, wherein said non-pregnant, pre-menopausal female is preferably of child-bearing potential. This combination has a significantly improved safety profile with concomitant efficacy. 4,4′-[fluoro-(1-H-1,2,4-triazol-1-yl)methylene]bisbenzonitrile is a potent and selective inhibitor of aromatase. The IC₅₀ and Kᵢ values for aromatase inhibition were determined in the microsomal fraction of human placenta and showed that 4,4′-[fluoro-(1-H-1,2,4-triazol-1-yl)methylene]bisbenzonitrile is a competitive inhibitor with an IC₅₀ of approximately 6.2 nM (Batzl-Hartmann et al, “Pharmacological profile of CGP47645, a new non-steroidal aromatase inhibitor with a long duration of action”, XVI International Cancer Congress, Pages 3041-3047, 1994). Toxicologic studies of the compound in female and male dogs showed that there was no consistent difference in exposure (AUC and Cₘₐₓ) between male and female dogs. Tₘₐₓ values were ranging from 1 h to 24 hrs post dose. Generally, the inter-animal variability in Cₘₐₓ levels was small. In general, following weekly oral dosing of the compound for 4 or 22 weeks, the mean plasma exposure to the compound was similar to that observed after a single dose at all dose levels tested, indicating there is no drug accumulation. An increase in exposure (AUC and Cₘₐₓ) was generally proportional to the dose increase for male and female dogs after single and multiple doses of the compound at all dose levels tested. Furthermore, measurement of testosterone levels in the serum of male dogs after 1, 4 and 12 weeks of dosing showed dramatically elevated testosterone levels at all dose levels demonstrating the potential of the compound.

In humans, 4,4′-[fluoro-(1-H-1,2,4-triazol-1-yl)methylene]bisbenzonitrile was initially studied in a single, ascending dose protocol in human female volunteers to assess safety and tolerability, as well as an ¹⁴C-4,4-[fluoro-(1-H-1,2,4-triazol-1-yl)methylene]bisbenzonitrile ADME study to determine tissue half-life of the drug. The first study showed that the median Tₘₐₓ occurred within 1 hour of ingestion, and that the half-life was directly dose dependent, with shorter half-lives at the lower doses.

Human pharmacokinetic (PK) and pharmacodynamic (PD) studies of marketed aromatase inhibitors in premenopausal women have shown that even doses 20× greater than those prescribed for postmenopausal women with breast cancer do not fully suppress endogenous estrogen production. However studies with the 4,4′-[fluoro-(1-H-1,2,4-triazol-1-yl)methylene]bisbenzonitrile in both females and males with obesity-associated hypogonadotropic hypogonadism demonstrated effects on the inhibition of aromatase-testosterone-estradiol pathway at doses as low as 0.003 mg and blood levels below the IC₅₀ of the aromatase enzyme.

The study of the 4,4′-[fluoro-(1-H-1,2,4-triazol-1-yl)methylene]bisbenzonitrile, for the treatment of endometriosis in non-pregnant, pre-menopausal females shows the safety of this treatment at low doses of the compound in a new dosage regimen for the target patient population.

The COC is used to ensure that the patient does not become pregnant whilst the 4,4′-[fluoro-(1-H-1,2,4-triazol-1yl)methylene]bisbenzonitrile is therapeutically active in said patient. This is an important safety feature of the present invention as 4,4′-[fluoro-(1-H-1,2,4-triazol-1-yl)methylene]bisbenzonitrile has been linked to teratogenic effects, and by removing the possibility of the patient falling pregnant whilst 4,4′-[fluoro-(1-H-1,2,4-triazol-1-yl)methylene]bisbenzonitrile is still therapeutically active in the patient, these teratogenic effects can thereby be avoided. Low doses of the 4,4′-[fluoro-(1-H-1,2,4-triazol-1-yl)methylene]bisbenzonitrile have been surprisingly found to effectively treat the symptoms of endometriosis by effectively inhibiting the estrogen synthesizing enzyme aromatase whilst having a short half-life such that the 4,4′-[fluoro-(1-H-1,2,4-triazol-1-yl)methylene]bisbenzonitrile's therapeutic activity effectively ends once the patient ends the course of the COC.

The rationale for administration of 4,4′-[fluoro-(1-H-1,2,4-triazol-1-yl)methylene]bisbenzonitrile being linked to menstrual cycle is as follows. Endometrial pain is most severe during the phase of the menstrual cycle. It is associated with high estradiol levels mid-cycle driving endometrial growth, including expansion of the ectopic endometriosis tissue with release of inflammatory cytokines, bleeding and irritation of pelvic nerves causing pain. On the other hand, estrogen increases pain thresholds (The Practice Committee of the American Society for Reproductive Medicine, Treatment of pelvic pain associated with endometriosis, Fertility and Sterility Vol. 90, S260-S269, November 2008). Thus, once the monthly estrogen-dependent expansion of tissue is completed during the first 3 weeks of the cycle, in some cases the dosing of the aromatase inhibitor may be reduced or stopped to enable systemic estrogen levels to rise.

Thus, in one embodiment, the the 4,4′-[fluoro-(1-H-1,2,4-triazol-1-yl)methylene]bisbenzonitrile is administered once every day, and the administration is discontinued or the dosing is reduced after 19, 20, 21 or 22 instances of daily administration.

In an alternative embodiment, the 4,4′-[fluoro-(1-H-1,2,4-triazol-1-yl)methylene]bisbenzonitrile is administered once every other day, and the administration is discontinued or the dosing is reduced after 10, 11 or 12 instances of every other day administration.

In a further embodiment, the 4,4′-[fluoro-(1-H-1,2,4-triazol-1-yl)methylene]bisbenzonitrile is administered once every 7 days, and the administration is discontinued after 3 instances of weekly administration.

Based on PK/PD analyses, it was found that the average estimated IC₄₀ and IC₉₀ of 4,4′-[fluoro-(1-H-1,2,4-triazol-1-yl)methylene]bisbenzonitrile for aromatase inhibition are 0.03 ng/mL and 0.27 ng/mL respectively (see Example 4). This concentration range defines the therapeutic window of effectiveness in respect of 4,4′-[fluoro-(1-H-1,2,4-triazol-1-y)methylene]bisbenzonitrile plasma concentration for aromatase inhibition. Evidence of suppression of aromatase inhibition was seen starting at doses of 0.003 mg with plasma levels demonstrated below IC₅₀ (see Example 3 and 4).

It was separately found that the concentration of 4,4′-[fluoro-(1-H-1,2,4-triazol-1-yl)methylene]bisbenzonitrile in human tissue (fat) where the aromatisation of steroid hormones to estrogens takes place is up to 10 times higher than in the blood.

It was also found that 4,4′-[fluoro-(1-H-1,2,4-triazol-1-yl)methylene]bisbenzonitrile and a continuously dosed COC may be co-administered safely without expecting any adverse effect on the aromatase inhibiting efficacy of 4,4′-[fluoro-(1-H-1,2,4-triazol-1-yl)methylene]bisbenzonitrile or the contraceptive efficacy of the COC (see example 5).

Surprisingly, there is a synergistic interaction between the COC and the low doses of the compound. The COC potentiates the effectiveness of the 4,4′-[fluoro-(1-H-1,2,4-triazol-1-yl)methylene]bisbenzonitrile, such that it is effective in the treatment of endometriosis at low doses of, for example, less than or equal to about 0.03 mg, such as about 0.01 mg, or as low as about 0.001 mg, where COC suppresses estrogen produced through the hypothamlamic-pituitary-gonadal pathway and 4,4′-[fluoro-(1-H-1,2,4-triazol-1-yl)methylene]bisbenzonitrile, through the tissue specific production in endometrial ectopic tissue and bathing pelvic fluid.

Preferably, the COC and 4,4′-[fluoro-(1-H-1,2,4-triazol-1-yl)methylene]bisbenzonitrile are presented such that therapeutic effectiveness of the 4,4′-[fluoro-(1-H-1,2,4-triazol-1-yl)methylene]bisbenzonitrile presented dosage form is concomitant with the COG dosage regimen. In other words, the in vivo levels of both the COC and 4,4′-[fluoro-(1-H-1,2,4-triazol-1-yl)methylene]bisbenzonitrile fall outside their respective therapeutic windows at the same time. This is preferably effected by matching the 4,4′-[fluoro-(1-H-1,2,4-triazol-1-yl)methylene]bisbenzonitrile therapeutic window to that of at least a single cycle of COC administration.

The compound may be mixed in a single formulation with the COC or may be formulated separately for simultaneous or sequential dosing.

The COC is preferably administered once daily for an n[28-day] cycle of treatment, where n is a positive integer multiplier, and preferably wherein n is between 1 and 3 inclusive.

The COC is a monophasic or multiphasic COC, preferably a monophasic COC.

The synthetic estrogen used in the COC is selected from estrogen, ethinyl estradiol or mestranol, preferably ethinyl estradiol.

Ethinyl estradiol can be administered or scheduled to administer at a dose between 15 and 50 μg/day, preferably 30 μg/day.

The synthetic progestogen used in the COC is selected from chlormadinone acetate, cyproterone acetate, desogestrel, dienogest, drospirenone, etynodiol diacetate, gestodene, levonorgestrel, norethisterone, norethisterone acetate, norgestimate, norgestrel, or nomegestrol acetate, preferably levonorgestrel, Levonorgestrel can be administered or scheduled to deliver at a dose between about 50 and about 250 μg/day, preferably about 150 μg/day.

Particularly preferred is a monophasic COC comprising about 30 μg/day of ethinyl estradiol and about 150 μg/day of levonorgestrel.

4,4′-[fluoro-(1-H-1,2,4-triazol-1-yl)methylene]bisbenzonitrile can be administered in doses from about 0.001 to about 0.1 mg doses, preferably from about 0.001 to about 0.06 mg doses, preferably from about 0.001 to about 0.03 mg doses, preferably from about 0.001 to about 0.01 mg doses, preferably from 0.001 to about 0.006 mg doses, preferably from about 0.001 to about 0.003 mg doses. These doses are preferably maintenance doses, as opposed to loading doses. For example, the compound can be administered in doses of about 0.001 mg, about 0.002 mg, about 0.003 mg, about 0.004 mg, about 0.005 mg, about 0.006 mg, about 0.007 mg, about 0.008 mg, about 0.009 mg, about 0.01 mg, about about 0.02 mg, about 0.03 mg, about 0.04 mg, about 0.05 mg, about 0.06 mg, about 0.07 mg, about 0.08 mg, about 0.09 mg, or about 0.1 mg, preferably doses of about 0.001 mg, about 0.003 mg, about 0.006 mg, about 0.01 mg or about 0.03 mg, further preferably doses of about 0.001 mg, about 0.003 mg or about 0.006 mg, even further preferably doses of about 0.001 mg.

The frequency of 4,4′-[fluoro-(1-H-1,2,4-triazol-1-yl)methylene]bisbenzonitrile's administration during an n[28-day] cycle of treatment is dose-dependent and may be administered with a frequency ranging from every day during the n[28-day] cycle to every 28 days during the n[28-day] cycle, from every day during the n[28-day] cycle to every 7 days during the n[28-day] cycle, or from every day during the n[28-day] cycle to every other day during the n[28-day] cycle.

For example, the frequency of the 4,4′-[fluoro-(1-H-1,2,4-triazol-1-yl)methylene]bisbenzonitrile's administration during an n[28-day] cycle of treatment can be every day during the n[28-day] cycle, every other day during the n[28-day] cycle, every 7 days during the n[28-day] cycle or every 28 days during the n[28-day] cycle, preferably every day, every other day or every 7 days during the n[28-day] cycle, further preferably every day or every other day during the n[28-day] cycle, even further preferably every day during the n[28-day] cycle.

Preferably, 4,4′-[fluoro-(1-H-1,2,4-triazol-1-yl)methylene]bisbenzonitrile is administered once every 28 days during the n[28-day] cycle for doses of about 0.05 mg to about 0.1 mg, preferably about 0.05 mg to about 0.075 mg.

Preferably, 4,4′-[fluoro-(1-H-1,2,4-triazol-1-yl)methylene]bisbenzonitrile is administered once every 7 days during the n[28-day] cycle for doses of about 0.009 mg to about 0.05 mg, preferably about 0.009 mg to about 0.03 mg.

Preferably 4,4′-[fluoro-(1-H-1,2,4-triazol-1-yl)methylene]bisbenzonitrile is administered once every other day during the n[28-day] cycle for doses of about 0.004 mg to about 0.009 mg, preferably about 0.004 mg to about 0.006 mg.

Preferably, 4,4′-[fluoro-(1-H-1,2,4-triazol-1-yl)methylene]bisbenzonitrile is administered once every day during the n[28-day] cycle for doses of about 0.001 mg to 0.004 mg, preferably about 0.001 mg to about 0.002 mg.

The half-life of 4,4′-[fluoro-(1-H-1,2,4-triazol-1-yl)methylene]bisbenzonitrile is dependent upon the dose administered to the patient, and in the present invention the dose is preferably tailored such that the compound has a half-life of less than or equal to 28 days. Alternatively less than or equal to 7 days. Alternatively less than or equal to every other day. Alternatively, less than or equal to a day.

The first dose of a given cycle is administered on the first day of said cycle, unless a loading dose is used. When a loading dose is used, the loading dose comprises a higher dose of the compound than the dose that will be repeatedly administered to the patient during the n[28-day] cycle of treatment (also known as a maintenance dose). The loading dose is to be administered to the patient at the at the beginning of a given cycle, whilst the frequency of subsequent maintenance doses remains dependent on the dose amount of the subsequent maintenance doses.

The loading dose is preferably 2 to 50 times the maintenance dose, more preferably 3 to 25 times the maintenance dose, more preferably 5 to 10 times the maintenance dose. For example, the loading dose may be administered to the patient in about 0.01 to about 0.1 mg doses, preferably about 0.01 to about 0.06 mg doses, preferably about 0.01 to about 0.03 mg doses. In each of these cases, the corresponding maintenance dose is lower than the loading dose.

The loading dose may be required in order to bring the blood-plasma concentration of the compound up to a level of therapeutic activity such that therapeutic activity can be maintained with subsequent doses. The loading dose is preferably used when the maintenance dose is less than or equal to about 0.06 mg, preferably less than or equal to about 0.03 mg, more preferably less than or equal to about 0.01 mg.

At the end of an n[28-day] period, levels of 4,4′-[fluoro-(1-H-1,2,4-triazol-1-yl)methylene]bisbenzonitrile will be depleted such that should the patient become pregnant immediately after the n[28 days], teratogenic effects of 4,4′-[fluoro-(1-H-1,2,4-triazol-1-yl)methylene]bisbenzonitrile will be avoided.

For the cases where 4,4′-[fluoro-(1-H-1,2,4-triazol-1-yl)methylene]bisbenzonitrile is mixed in a single formulation with the COC, the number of mixed formulations in a given 28-day period is determined by the dose of 4,4′-[fluoro-(1-H-1,2,4-triazol-1-yl)methylene]bisbenzonitrile. The COO alone is preferably taken on all days that a dose of 4,4′-[fluoro 1-H-1,2,4-triazol-1-yl ethylene]bisbenzonitrile is not required.

For the cases where 4,4′-[fluoro-(1-H-1,2,4-triazol-1-yl)methylene]bisbenzonitrile and COC are formulated separately for simultaneous or sequential dosing, the COC is taken on all days and 4,4′-[fluoro-(1-H-1,2,4-triazol-1-yl)methylene]bisbenzonitrile is taken only on the days required to achieve an effective dose for treating endometriosis. The days that 4,4′-[fluoro-(1-H-1,2,4-triazol-1-yl)methylene]bisbenzonitrile should be taken is set by the dose of the compound, and is administered either simultaneously with the COC or immediately before/after the COC.

The n[28-day] cycle of treatment occurs over an m[28-day] period of time where m is a positive integer multiplier and where m is at least 1, 2 or 3, such as at least 6, at least 12 or at least 24. More than one n[28-day] cycle of treatment may occur in each year (i.e. when m=12). Each n[28-day] cycle of treatment may be punctuated by one or more cycles of non-treatment, i.e., non-administration of COC and/or 4,4′-[fluoro-(1-H-1,2,4-triazol-1-yl)methylene]bisbenzonitrile.

4,4′-[fluoro-(1-H-1,2,4-triazol-1-yl)methylene]bisbenzonitrile and COC may be provided in various formulations such as parentally (e.g. aqueous or oily suspensions) or orally (e.g., tablets, powders, capsules, granules, aqueous or oily suspensions). Preferably, the compound is provided in an orally available formulation to be administered according to the described dosing regimen. However, slow release formulation or depot or transdermal formulations could also be used to administer the compound.

In preparation of formulations according to the invention, one or more pharmaceutically acceptable excipients, such as inert pharmaceutically acceptable carriers, may optionally be used in combination with the active component(s) of the formulations, which can either be solid or liquid. Solid form preparations include cachets, capsules, dispersible granules, powders and tablets.

The term formulation is intended to include the mixture of the active component(s) with encapsulating material as a carrier providing a capsule in which the active compound (with or without other carriers) is surrounded by a carrier which is thus in association with it. Similarly, cachets are included. Tablets, powders, cachets, and capsules can be used as solid dosage forms suitable for oral administration.

The pharmaceutical formulation can be in unit dosage form. In such form, the composition is divided into unit doses containing appropriate quantities of the active component(s). The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

According to another aspect of the present invention, there is provided a multi-phase combination preparation comprising n[28-day] dosage units of the 4,4-[fluoro-(1-H-1,2,4-triazol-1-yl)methylene]bisbenzonitrile and COC dosage regimen herein defined, where n is a positive integer between 1 and 3 inclusive. Each of these dosage units comprise either a combination of the compound mixed in a single formulation with the COC or formulated separately for simultaneous or sequential dosing, or the COC alone, as determined by the dose of the compound.

According to another aspect of the present invention, there is provided a kit of parts comprising: (i) a multi-phase combination preparation comprising n[28-day] dosage units of the such that each dosage unit comprises the 4,4″-[fluoro-(1-H-1,2,4-triazol-1-yl)methylene]bisbenzonitrile and combined oral contraceptive in a combined form, separately, or the combined oral contraceptive alone and COC dosage regimen herein defined, where n is an integer between 1 and 3; together with (ii) instructions how to administer said dosage regimen for the treatment of a non-pregnant, pre-menopausal female with endometriosis. Each of these dosage units comprise either a combination of the compound mixed in a single formulation with the COC or formulated separately for simultaneous or sequential dosing, or the COC alone, as determined by the dose of the compound.

### EXAMPLES

### Example 1: Preparation of 4,4′-[fluoro-(1-H-1,2,4-triazol-1-yl)methylene]bisbenzonitrile

The following example describes a method for the synthesis of 4,4′-[fluoro-(1-H-1,2,4-triazol-1-yl)methylene]bisbenzonitrile (also known as 4-[α-4-Cyanophenyl)-α-fluoro-1-1,2,4-triazolyl)-methyl]-benzonitrile or CGP47645) as disclosed within Lang et al., U.S. Pat. No. 5,637,605:

A solution of 0.8 mmol of potassium hexamethyldisilazane in 1.6 ml of toluene is diluted with 5 ml of THF and, after cooling to −78° C., a solution of 190 mg of 4-[α-(4-cyanophenyl)-1-(1,2,4-triazolyl)methyl]-benzonitrile (see EP-A-236 940, Ex. 20a) in 3 ml of THF is added thereto. After stirring for 1 hour at the same temperature, there are added dropwise to the dark-red solution 301 mg of N-fluoro-dimethylsaccharinsultam in 3 ml of THF. After a further 1.5 hours at −78° C., the reaction mixture is heated to room temperature within 1 hour and poured onto a saturated solution of ammonium chloride in water and then extracted with methylene chloride. Drying over magnesium chloride and concentration of the solvent by evaporation yields the crude product which is purified by means of flash-chromatography (SiO2, hexane/ethyl acetate 9:1, 4:1 to 1:1). TLC (SiO2, CHCl3/methanol 9:1, Rf=0.85); IR (KBr): 2220 cm−1; 1H-NMR (CDCl3): δ (ppm) =7.46 and 7.76 (8H, m), 8.07 (1H, s), 8.16 (1H, s).

All disclosure relevant to the preparation of 4-[α-4-Cyanophenyl)-α-fluoro-1-1,2,4-triazolyl)-methyl]benzonitrile described in Lang et al., U.S. Pat. No. 5,376,669 is hereby incorporated by reference herein.

The above paragraph refers to EP-A-236 940, Ex. 20a. The U.S. equivalent to EP-236 940 is Bowman, U.S. Pat. No. 4,749,713. Example 20 (a) of EP-A-236 940 (U.S. Pat. No. 4,749,713) states that 4-[1-(1,2,4-Triazolyl)-methyl]-benzonitrile is reacted with potassium tert-butoxide and 4-fluorobenzonitrile according to the procedure in Example 2 of U.S. Pat. No. 4,749,713 to yield 4-[α-(4-cyanophenyl)-1-(1,2,4-triazolyl)-methyl]benzonitrile, m.p. 181° C.-183° C.

The procedure of Example 2 of U.S. Pat. No. 4,749,713 provides that: A suspension of potassium tert-butoxide (61.6 g) in dimethylformamide (500 mL) is stirred and cooled to −10° C. (ice-salt bath), and a solution of 4-(1-imidazolylmethyl)-benzonitrile (45.6 g) in dimethylformamide (250 mL) is added so that the reaction temperature remains below 0° C. The resulting solution is stirred at 0° C. for 0.5 hour and then a solution of 4-fluorobenzonitrile (38.3 g) in dimethylformamide (100 mL) is added while keeping reaction temperature below 5° C. After 0.75 hour, the reaction mixture is neutralized to pH 7 by addition of sufficient 3N hydrochloric acid and the bulk of the solvents are then removed under reduced pressure. The residue is diluted with water (500 mL) and the crude product is extracted into ethyl acetate (3×200 mL). The combined extracts are then extracted with 3N hydrochloric acid (3×150 mL) and, after washing the latter acid extracts with ethyl acetate (100 mL), the solution is made basic (pH 8) with 6N ammonium hydroxide and the product is again extracted into ethyl acetate (3×150 mL). The combined extracts are dried (MgSO4), decolorized by treatment with charcoal, and then evaporated to give crude 4-[α-(4-cyanophenyl)-1-imidazolylmethyl]-benzonitrile as an oil. This material is dissolved in isopropanol (250 mL) and the warm solution is stirred with succinic acid (14.4 g). Upon dilution with diethyl ether (100 mL) and stirring at ambient termperature, the hemi-succinate salt separates. The salt is filtered off, washed with a little cold isopropanol and then air dried to afford 4-[α-(4-cyanophenyl)-1-imidazolylmethyl]-benzonitrile hemisuccinate, m.p. 149° C.-150° C. The hemifumarate salt has nip, 157° C.-158° C.

All disclosure relevant to the preparation of 4-[α-(4-cyanophenyl)-1-(1,2,4-triazolyl)-methyl]benzonitrile described in Bowman, U.S. Pat. No. 4,749,713 is hereby incorporated by reference herein.

### Example 2: Low-Dose Capsule Formulations of 4,4′-[fluoro-(1-H-1,2,4-triazol-1-yl)methylene]bisbenzonitrile (CGP47645)

CGP47645 containing hard gelatine capsules are prepared by the following process: The required excipients, in the respective amounts to yield the final composition as indicated in Table 1 below, and the appropriate amount of CGP47645 drug substance are weighed.

Then, approximately 50% of corn starch is filled into suitable container, the drug substance is added, followed by the remaining 50% of corn starch to get a sandwich of drug substance between two layers of maize starch. Blending and sieving this mixture yields the drug substance (DS) premix.

The remaining excipients (microcrystalline cellulose, spray-dried lactose, sodium starch glycolate, and colloidal silicon dioxide [Aerosil® 200]) are mixed and sieved and transfer into a suitable container. Then the DS premix is added into container containing the sieved excipients and the mixture is blended together. Finally, pre-sieved Magnesium stearate is added to the blend containing the DS and this mixture is blended again to yield the final blend. The final blend is filled into hard gelatin capsules.

All the excipients comply with the requirements of the applicable compendial monographs (Ph. Eur., NF). The hard gelatine capsules are packaged in HDPE bottles with aluminum induction seal equipped with child-resistant screw-cap closures.

The final dosage form is a hard gelatine capsule containing a white to yellowish powder in a pink opaque capsule, size 1 or 3.

TABLE 1
composition of the CGP47645 hard gelatin capsule of 0.1 mg,
0.5, 1 mg and 10 mg strength.

| | Amount per capsule (mg) | | | | |
|---|---|---|---|---|---|
| Ingredient | 0.1 mg¹ | 0.1 mg² | 0.5 mg¹ | 1 mg² | 10 mg² |
| Capsule content | | | | | |
| CGP47645 | 0.1 | 0.1 | 0.5 | 1.0 | 10.0 |
| Lactose monohydrate | 96.0 | 192.0 | 96.0 | 192.0 | 175.5 |
| Cellulose, | 30.0 | 60.0 | 30.0 | 60.0 | 50.0 |
| microcrystalline | | | | | |
| Corn Starch | 14.15 | 28.4 | 13.75 | 27.5 | 40.0 |
| Sodium starch | 7.5 | 15.0 | 7.5 | 15.0 | 15.0 |
| glycolate (Type A) | | | | | |
| Magnesium Stearate | 1.5 | 3.0 | 1.5 | 3.0 | 3.0 |
| Silica, colloidal | 0.75 | 1.5 | 0.75 | 1.5 | 1.5 |
| anhydrous | | | | | |
| Capsule fill weight | 150.0 | 300.0 | 150 | 300.0 | 295.0 |
| Empty capsule shell | | | | | |
| Capsule shell | 48.0 | 76.0 | 48.0 | 76.0 | 76.0 |
| Total capsule weight | 198.0 | 376.0 | 198.0 | 376.0 | 371.0 |

¹Filled in size 3 capsules;
²Filled in size 1 capsules

### Example 3: Single Ascending Dose Study of 4,4′-[Fluoro-(1-H-1,2,4-triazol-1-yl)methylene]bisbenzonitrile (CGP47645)

This was a randomized, double-blind, placebo- and active-controlled single ascending dose study in pre- and post-menopausal women to assess the safety and tolerability, PK and PD effects of single doses of 4,4′-[Fluoro-(1-H-1,2,4-triazol-1-yl)methylene]bisbenzonitrile (CGP47645). There were 8 cohorts of 8 post-menopausal subjects randomized 6:2, CGP47645:placebo, who received single doses of CGP47645 beginning at the dose of 0.01 mg and carried through 20 mg, which reached the limit of the toxicology exposure coverage. Patients received either 0.1 mg, 1 mg, and 10 mg drug substance containing hard gelatin capsules or appropriate matching placebo capsules. For the lowest two dosing cohorts, 0.1 mg drug containing capsules were used for reconstituting the CGP47645 oral solutions for dosing the 0.01 and 0.03 dosing strength (Cohort 1 and 2).

A minimal toxic dose (MTD) was not reached. A single cohort of 8 pre-menopausal subjects without childbearing potential (Cohort No. 9) received CGP47645 0.1 mg or placebo, randomized 6:2, and one last cohort received letrozole 2.5 mg as an internal positive control cohort for the PD measurements. Table 3 presents the PK parameters based on analysis of the concentration-time profile obtained from this study.

TABLE 2
CGP47645 Pharmacokinetics in Post- & Pre-menopausal women

| | | | | | | AUC(0-tₗₐₛₜ) | | | |
|---|---|---|---|---|---|---|---|---|---|
| | | | Cₘₐₓ (ng/mL) | | | (ng*hr/mL) | | T_{1/2} (days) | |
| Dose | Cohort | | | CV | Tₘₐₓ (hr) | | CV | | CV |
| (mg) | No. | Size | Mean | (%) | Median | Mean | (%) | Mean | (%) |
| 0.01 | 1 | (n = 5) | 0.2 | 21.7 | 1 | 1.4 | 53.2 | 2.3 | 127.5 |
| 0.03 | 2 | (n = 6) | 0.4 | 18.7 | 0.6 | 24.1 | 34.0 | 16.5 | 36.0 |
| 0.1 | 3 | (n = 6) | 1.8 | 13.4 | 1 | 123.1 | 10.7 | 18.2 | 10.9 |
| 0.3 | 4 | (n = 6) | 5.1 | 14.1 | 1 | 605.1 | 49.0 | 23.5 | 19.9 |
| 1 | 5 | (n = 5) | 12.8 | 22.0 | 1 | 3201.9 | 37.2 | 22.4 | 38.5 |
| 3 | 6 | (n = 6) | 38.4 | 17.0 | 1 | 10053.0 | 16.7 | 25.0 | 8.4 |
| 10 | 7 | (n = 6) | 123.8 | 26.4 | 2 | 41745.5 | 17.3 | 27.3 | 17.6 |
| 20 | 8 | (n = 6) | 269.8 | 30.9 | 2 | 76731.6 | 11.4 | 26.9 | 16.5 |
| 0.1 | 9 | (n = 6) | 1.7 | 15.1 | 1 | 116.2 | 17.1 | 23.5 | 31.0 |
| 2.5 | Letrozole | (n = 8) | 33.5 | 27.0 | 1 | 1667.7 | 40.8 | 2.9 | 40.7 |

CGP47645 exhibited dose proportional pharmacokinetics and a dose-dependent inhibition of estrone, estrone sulfate and estradiol, No differences in CGP47645 pharmacokinetics were observed between post- and pre-menopausal women. CGP47645 is rapidly absorbed with a Tmax of 0.5-2 hrs; the median Tmax occurred within 1 hour of ingestion. Both Cₘₐₓ & AUC increased in a dose-proportional manner. CGP47645 exhibited low inter-subject variability of 10-30% and completely unexpected long half-life in the range of 23 to 27 days.

In postmenopausal women the study showed evidence of efficacy in PD parameters with estrone suppression at least equal to letrozole already at doses of 0.1 mg and 0.3 mg. In postmenopausal women, the lowest single dose at which transient estrogen suppression was seen was 0.01 mg; and the lowest single dose at which maximal estrogen suppression was observed in post-menopausal women, using chemiluminescence or radioimmunoassay, was 0.1 mg.

### Example 4: Low Dose Study of 4,4′-[Fluoro-(1-H-1,2,4-triazol-1-yl)methylene]bisbenzonitrile (CGP47645) in Men

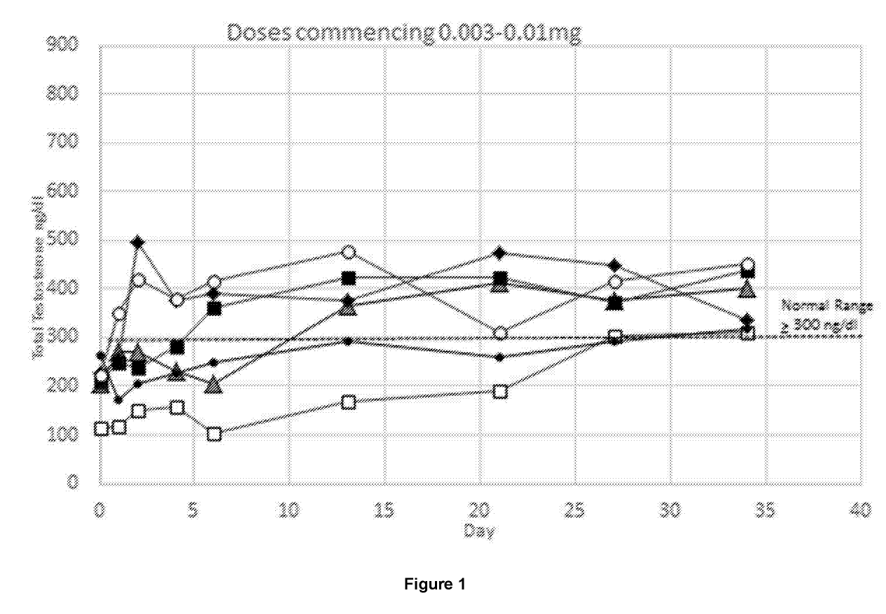

Obese men with low testosterone levels (less than 300 ng/ml) due to the activity of the high levels of aromatase in the adipose tissue were given doses of CGP47645 starting as low as 0.003 mg and then titrated up. By inhibiting the aromatase enzyme, CGP47645 led to an increase in circulating levels by reducing the conversion of testosterone to estradiol. This study confirmed that doses of CGP47645 between 0.003 and 0.01 mg are biologically active in inhibition of aromatisation pathway as evidenced in FIG. 1.

Based on PK/PD analyses, the average estimated IC₅₀ and IC₉₀ of CGP47645 for aromatase inhibition are 0.03 ng/mL and 0.27 ng/mL respectively.

### Example 5: Single Dose Study of the Efficacy of 4,4′-[Fluoro-(1-H-1,2,4-triazol-1-yl)methylene]bisbenzonitrile (CGP47645) and COC when Used in Combination in Women

A randomized, double blind, placebo controlled, parallel group study to assess whether a single 1.0 mg oral dose of CGP47645 in combination with the combined oral contraceptive is safe and does not affect the pharmacokinetic properties and the expected efficacy of the combined oral contraceptive in pregnancy prevention.

The study consisted of a 49-day screening period and four consecutive 28-day (menstrual) cycles.

For the combined oral contraceptive, 30 μg of ethinyl estradiol and 0.15 mg of levonorgestrel (Seasonique®) was used. Seasonique® is an oral contraceptive designed and approved for daily administration. The 84 blue-green tablets (30-μg ethinyl estradiol and 0.15 mg levonorgestrel) are packaged with seven yellow tablets containing 10-μg ethinyl estradiol to eliminate the hormone free interval.

Arithmetic mean plasma concentration time profile for CGP47645 administered as detailed above in 15 healthy, premenopausal women showed pharmacokinetic parameters similar to those obtained in the single ascending dose study where BGS649 was administered to healthy post-menopausal women without COG co-administration (Example 3).

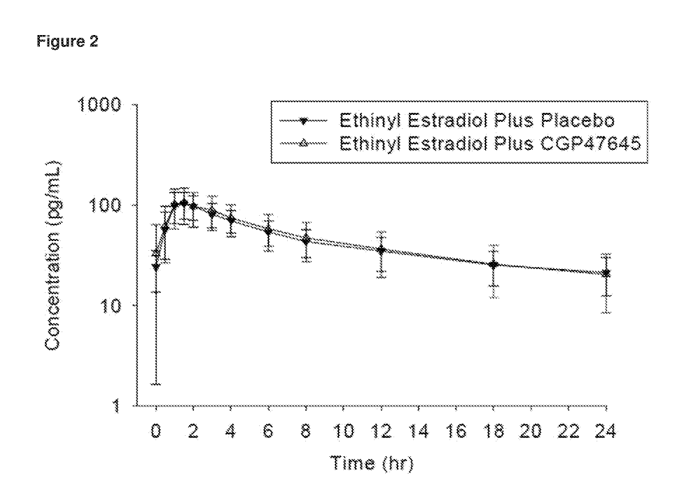

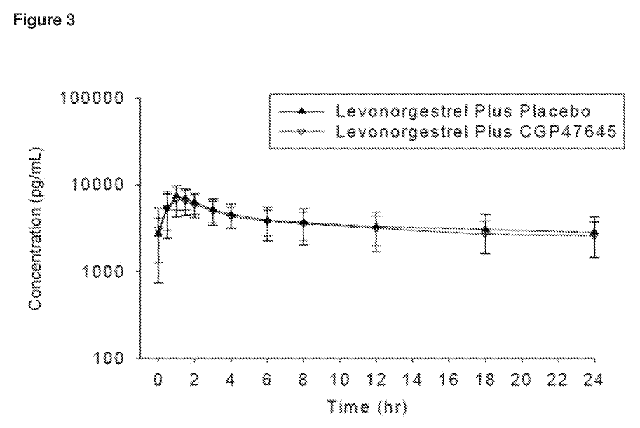

Mean steady state plasma concentration time profiles for ethinyl estradiol (30 μg) and levonogestrel (0.15 mg) on Day 29 from surgically sterile healthy premenopausal women administered COO with placebo (N=14) or 1 mg GGP47645 (N=15) are depicted in FIGS. 2 and 3 respectively. The pharmacokinetic profiles of both ethinyl estradiol and levonorgestrel when administered with either placebo or GGP47645 appear similar.

GGP47645 and a continuously dosed COC containing ethinyl estradiol and levonorgestrel may be co-administered safely without expecting any adverse effect on the contraceptive efficacy of the COC.

What is claimed is:

1. A dosage regimen for the treatment of endometriosis in a non-pregnant, pre-menopausal female, comprising the administration to a patient in need thereof 4,4′-[fluoro-(1-H-1,2,4-triazol-1-yl)methylene]bisbenzonitrile and a combined oral contraceptive, wherein the 4,4′-[fluoro-(1-H-1,2,4-triazol-1-yl)methylene]bisbenzonitrile is administered in doses of about 0.001 mg to about 0.1 mg, wherein the combined oral contraceptive is administered once daily for an n[28-day] cycle of treatment, wherein n is a positive integer multiplier, wherein n is between 1 and 3 inclusive, and wherein the 4,4′-[fluoro-(1-H-1,2,4-triazol-1-yl)methylene]bisbenzonitrile is administered once every day, once every other day, once every 7 days or once every 28 days during the n[28-day] cycle of treatment.

2. The dosage regimen according to claim 1, wherein the 4,4′-[fluoro-(1-H-1,2,4-triazol-1-yl)methylene]bisbenzonitrile is administered once every day during the n[28-day] cycle of treatment for doses of about 0.001 mg to about 0.004 mg.

3. The dosage regimen according to claim 1, wherein the 4,4′-[fluoro-(1-H-1,2,4-triazol-1-yl)methylene]bisbenzonitrile is administered once every other day during the n[28-day] cycle of treatment for doses of about 0.004 mg to about 0.009 mg.

4. The dosage regimen according to claim 1, wherein the 4,4′-[fluoro-(1-H-1,2,4-triazol-1-yl)methylene]bisbenzonitrile is administered once every 7 days during the n[28-day] cycle of treatment for doses of about 0.009 mg to about 0.05 mg.

5. The dosage regimen according to claim 1, wherein the 4,4′-[fluoro-(1-H-1,2,4-triazol-1-yl)methylene]bisbenzonitrile is administered once every 28 days during the n[28-day] cycle of treatment for doses of about 0.05 mg to about 0.1 mg.

6. The dosage regimen according to claim 1, wherein the plasma concentration of 4,4[-fluoro-(1-H-1,2,4-triazol-1-yl)methylene]bisbenzonitrile in the non-pregnant, pre-menopausal female remains between 0.03 ng/mL and 0.27 ng/mL for the duration of the n[28-day] cycle of treatment and drops below 0.03 ng/mL when the cycle of treatment ends.

7. The dosage regimen according to claim 1, wherein n is 1.

8. The dosage regimen according to claim 1, wherein n is 2.

9. The dosage regimen according to claim 1, wherein n is 3.

10. The dosage regimen according to claim 1, wherein the 4,4′-[fluoro-(1-H-1,2,4-triazol-1-yl)methylene]bisbenzonitrile is mixed in a single formulation with the combined oral contraceptive or is formulated separately for simultaneous or sequential dosing.

11. The dosage regimen according to claim 1, wherein the combined oral contraceptive is monophasic or multiphasic.

12. The dosage regimen according to claim 11, wherein the combined oral contraceptive is monophasic.

13. The dosage regimen according to claim 1, wherein the combined oral contraceptive comprises a synthetic estrogen selected from estrogen, ethinyl estradiol, and mestranol.

14. The dosage regimen according to claim 13, wherein the synthetic estrogen is ethinyl estradiol.

15. The dosage regimen according to claim 14, wherein the dosage regimen comprises ethinyl estradiol administered at 15-50 μg/day.

16. The dosage regimen according to claim 15, wherein the dosage regimen comprises ethinyl estradiol administered at 30 μg/day.

17. The dosage regimen according to claim 1, wherein the combined oral contraceptive comprises a synthetic progestogen selected from chlormadinone acetate, cyproterone acetate, desogestrel, dienogest, drospirenone, etynodiol diacetate, gestodene, levonorgestrel, norethisterone, norethisterone acetate, norgestimate, norgestrel, and nomegestrol acetate.

18. The dosage regimen according to claim 17, wherein the synthetic progestogen is levonorgestrel.

19. The dosage regimen according to claim 18, wherein the dosage regimen comprises levonorgestrel administered at 50-250 μg/day.

20. The dosage regimen according to claim 19, wherein the dosage regimen comprises levonorgestrel administered at 150 μg/day.

21. The dosage regimen according to claim 1, wherein a loading dose of the 4,4′-[fluoro-(1-H-1,2,4-triazol-1-yl)methylene]bisbenzonitrile is administered at the beginning of the n[28-day] cycle of treatment and maintenance doses of the 4,4′-[fluoro-(1-H-1,2,4-triazol-1-yl)methylene]bisbenzonitrile are administered for the remainder of the n[28-day] cycle of treatment, and wherein the maintenance doses are lower than the loading dose.

22. The dosage regimen according to claim 1, wherein the non-pregnant, pre-menopausal female is of child-bearing potential.

23. The dosage regimen according to claim 1, wherein the 4,4′-[fluoro-(1-H-1,2,4-triazol-1-yl)methylene]bisbenzonitrile and the combined oral contraceptive are administered parentally or orally.

24. The dosage regimen according to claim 23, wherein the 4,4′-[fluoro-(1-H-1,2,4-triazol-1-yl)methylene]bisbenzonitrile and the combined oral contraceptive are administered as solid form preparations.

25. The dosage regimen according to claim 1, wherein the 4,4′-[fluoro-(1-H-1,2,4-triazol-1-yl)methylene]bisbenzonitrile is administered once every day, and the administration is discontinued or the dosing is reduced after 19, 20, 21 or 22 instances of daily administration.

26. The dosage regimen according to claim 1, wherein the 4,4′-[fluoro-(1-H-1,2,4-triazol-1-yl)methylene]bisbenzonitrile is administered once every other day, and the administration is discontinued or the dosing is reduced after 10, 11 or 12 instances of every other day administration.

27. The dosage regimen according to claim 1, wherein the 4,4′-[fluoro-(1-H-1,2,4-triazol-1-yl)methylene]bisbenzonitrile is administered once every 7 days, and the administration is discontinued after 3 instances of weekly administration.

28. A multi-phase combination preparation for use in the dosage regimen of claim 1, wherein the multi-phase combination preparation comprises n[28-day] dosage units consistent with the dosage regimen such that each dosage unit comprises the 4,4′-[fluoro-(1-H-1,2,4-triazol-1-yl)methylene]bisbenzonitrile and combined oral contraceptive in a combined form, separately, or the combined oral contraceptive alone.

29. A kit comprising the multi-phase preparation according to claim 28, wherein the kit further comprises instructions on how to administer the dosage regimen for the treatment of a non-pregnant, pre-menopausal female with endometriosis.
